(19) United States
(12) Patent Application Publication
(10) Pub. No.: US 2003/0119051 A1
(43) Pub. Date: Jun. 26, 2003

(54) SACCHARIDE LIBRARY

(76) Inventors: MANFRED WIESSLER, HEIDELBERG (DE); CHRISTIAN KLIEM, HEPPENHEIM (DE); WALTER MIER, ZWINGENBERG (DE); STEFAN MENZLER, WIESBADEN (DE)

Correspondence Address: ALBERT P HALLUIN, HOWREY & SIMON, 1299 PENNSYLVANIA AVENUE NW, BOX NO 34, WASHINGTON, DC, 200042402

(21) Appl. No.: 09/284,776

(22) PCT Filed: Oct. 15, 1997
(86) PCT No.: PCT/DE97/02372

(30) Foreign Application Priority Data
Oct. 16, 1996 (DE) 196 42 751.7

(51) Int. Cl. C12Q 1/70; G01N 33/53; C12P 21/04; G01N 33/543

(52) U.S. Cl. 435/7.1; 436/518; 435/5

(57) ABSTRACT

The present invention relates to a saccharide library with different saccharide-containing molecules, in which each of the molecules comprises a nuclear molecule with at least two functional groups and at least two saccharides. The invention also relates to the production of such a library and its use.

The present invention relates to a saccharide library, processes for the production thereof and its use.

For some time, it has been considered to provide active substances, e.g. therapeutic agents, on a saccharide basis. This applies particularly when the active substances shall be agonists of cell receptors and antagonists thereof, respectively. However, it has been extremely difficult so far to provide active substances on a saccharide basis, i.e. to find those which react precisely with target proteins, e.g. receptors.

Therefore, it is the object of the present invention to provide a product by means of which it is possible to find active substances on a saccharide basis.

According to the invention this is achieved by the subject matters defined in the claims.

Thus, the subject matter of the invention relates to a saccharide library having various saccharide-containing molecules, each of the saccharide-containing molecules comprising a nuclear molecule having at least two functional groups and at least two saccharides.

The above expression “saccharide library” stands for a plurality of, e.g. at least 6, preferably at least 20, more preferably at least 50, and most preferably at least 100, different saccharide-containing molecules. These molecules can be present in unbound form or bound to a carrier. The suitable carriers are all matrices that are used in solid-phase chemistry, such as solid phases on the basis of polystyrene, polyethylene glycol, kieselguhr, CPC (controlled pore ceramics), cellulose and glass.

The above expression “nuclear molecule having at least two functional groups” comprises aliphatic compounds having at least two, particularly 3, 4, 5 or 6, functional groups, e.g. hydroxy groups, amino groups, carboxylic acid groups, metallo-organic groups and/or halide groups. The functional groups may be the same or differ from one another. Examples of nuclear molecules are cyclic aliphatic compounds. Representatives thereof are C₆ cycloalkanes, such as trihydroxycycloalkanes, e.g. 1,3,5-trihydroxycycloalkanes, particularly 1,3,5-trihydroxycyclohexane, inositols, particularly myo-inositol, and C₅ cycloalkanes, such as tri- and tetrahydroxycyclopentanes, as well as derivatives thereof. In addition, nuclear molecules are heterocyclic hydroxy compounds. Moreover, nuclear molecules are aliphatic amines, such as triamines, particularly methylene triamine, and pentaerythritols. Particularly preferred nuclear molecules are shown in FIG. 1. Steroids, cholic acid methyl ester and saccharides are no nuclear molecules within the meaning of this invention.

The above expression “saccharide” comprises any kinds of saccharides in all stereoisomeric and enantiomeric forms, particularly monosaccharides, e.g. pentoses and hexoses, such as α- and β-D-glucose and α- and β-D-mannose, as well as disaccharides, trisaccharides and oligosaccharides. Within the meaning of the above-mentioned saccharides it is also possible to bind to the nuclear molecule inositols, very particularly optically active derivatives of myoinositol and quebrachitol, e.g. from galactinols, from both vegetable sources, such as sugar beets, and milk products, or derivatives obtained by enzymatic enantiomer separation. Furthermore, saccharides are glycoconjugates. They can be conjugates of saccharides with peptides, heterocycles and other carbohydrates. An example of glycoconjugates is Z1-Z10, a mixture of 10 glycoconjugates. The Z1-Z10 compounds are naturally occurring glycopeptides, glycoproteins and lipopolysaccharides. All of these compounds are of great biological interest because of the part they play in various immunological processes. An example thereof is

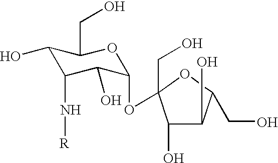

wherein R denotes amino acids, e.g. asparagic acid, lysine, glycine, alanine, etc., or fatty acids. Derivatives of the above saccharides, such as saccharides protected by protecting groups, e.g. benzyl, and/or saccharides modified by functional groups, such as amino groups, phosphate groups or halide groups are also considered to be saccharides. The above saccharides can occur naturally or be produced synthetically. A saccharide-containing molecule preferably has 3, 4, 5 or 6 saccharides. The saccharides may be equal or differ from one another. In the saccharide-containing molecule, several of the saccharides may be equal and one or several of the other saccharides may differ therefrom. For example, one saccharide may be a disaccharide, trisaccharide or oligosaccharide and the others are e.g. monosaccharide. This is referred to as a saccharide background library (cf. FIG. 3). The binding of the saccharides to the nuclear molecule can be made via the functional groups thereof. This is done preferably by forming an O-glycosidic bond.

In a preferred embodiment a spacer is present between the nuclear molecule and one to maximally all saccharides. Examples thereof are aliphatic compounds such as alkanes. The spacer can also be an unsaturated aliphatic compound. The spacer preferably has 3 to 10 C atoms. Furthermore, the spacer can be bound to the functional groups of the nuclear molecule and/or the saccharides. If several spacers are present, they may be equal or differ from one another.

A saccharide-containing molecule present in the library according to the invention preferably has an organic compound. The latter can be bound to the nuclear molecule and/or to one or several of the saccharides. Examples of organic compounds are alkanes having a functional group, e.g. a halogen, such as bromine, a hydroxy, azido and/or amino group, or alkenes, particularly with terminal double bond. The alkenes may also include the above functional groups. The above organic compound preferably has 3 to 10 C atoms. In addition, one or several of the organic compounds can be present. If several are present, they may be the same or differ from one another. By means of the organic compounds it is e.g. possible to bind the saccharide-containing molecule to a carrier and/or to bind dyes, magnetic particles and/or other components to the saccharide-containing molecule.

The components of the saccharide-containing molecules are shown as educts. However, in the saccharide-containing molecules they are present in derivatized form.

According to the invention a process for the production of the above-mentioned saccharide libraries is also provided. In this process, the individual components, i.e. nuclear molecules, saccharides, optionally linkers, optionally organic compound and optionally carriers are bonded covalently with one another.

For example, a nuclear molecule bound to a carrier is provided in which the functional groups have protecting groups. The protecting groups may be orthogonal protecting groups. These protecting groups distinguish themselves in that they can be cleaved separately (selectively), i.e. one after the other, from a molecule in the presence of other protecting groups, without these other protecting groups being influenced by the cleavage conditions. Examples of such protecting groups are acyl groups, such as benzoyl, acetyl and chloroacetyl, benzyl groups and silyl groups. The person skilled in the art knows how to cleave them selectively. One of these protecting groups is cleaved. Thereafter, reaction is carried out with a saccharide or a mixture of saccharides, so that the saccharides are bound to the functional group. Then, the next protecting group is cleaved selectively, and the reaction is repeated. In this connection, it is possible to use a new saccharide, a new mixture of saccharides or the saccharide or mixture of saccharides which were used in the preceding step. These reactions can be repeated until all desired functional groups of the nuclear molecule have a saccharide. Finally, the resulting saccharide-containing molecules can be split off the carrier and, if desired, the protecting groups optionally present at the saccharides can be split off. In this way, saccharide libraries according to the invention are obtained. If only one kind of saccharides are used as saccharides in the individual steps, only one kind of saccharide-containing molecules will be obtained. They can be mixed with saccharide-containing molecules differing therefrom to give a saccharide library. If in the above reaction mixtures of saccharides are used, a combination of various saccharide-containing molecules (=saccharide library) will be obtained. This can be shown by the example of a solid phase-linked inositol as follows:

| | | |
|---|---|---|
| 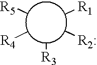 | | Solid phase to which inositol is bound; R₁—R₅: orthogonal protecting groups |
| A, B, C: | | 3 different saccharides which can be linked to the solid phase |
| | 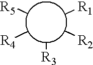 | |
| I. linkage | 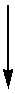 | 1. Selective cleavage of R₁2. linkage to a mixture of A, B and C |
| 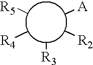 | 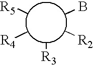 | 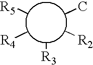 |
| II. linkage | 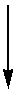 | 1. Selective cleavage of R₂2. linkage to a mixture of A, B and C |
| 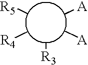 | 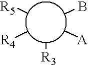 | 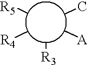 |
| 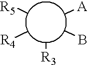 | 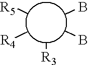 | 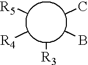 |
| 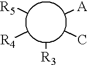 | 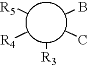 | 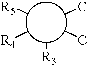 |

| | |
|---|---|
| III. | linkage |
| IV. | linkage |
| V. | linkage |

The number of differing library building blocks after 5 linkages (as shown above) then follows from the general valid formula:

Z=M^{F}

Z=number of differing library building blocks; M=number of differing saccharide species which are used as a mixture for the linkage to the central building block (here: 3 different monosaccharides); F=number of the functionalities of the central building block (OH—, NH₂— groups . . . , here: 5 OH groups).

Z=3⁵=243

As described above, e.g. monosaccharides can be bound to the nuclear molecule. They may be equal or differ from one another. One of these monosaccharides has a group, e.g. an acetyl group, which is capable of binding to another saccharide. A saccharide differing from the already bound saccharides is then bound to this site. Finally, the resulting saccharide-containing molecules can be split off the carrier and, if desired, the protecting groups optionally present at the saccharides can be cleaved. A saccharide background library can be obtained in this way.

The glycosidation of a nuclear molecule, as described in FIGS. 2-4, can be made chemically and enzymatically. In the latter case, use is made of the fact that glycosidases can transfer monosaccharides from activated donor saccharides (nitrophenyl glycosides, glycals, glycosyl fluorides, disaccharides, etc.) to suitable acceptors (transglycosidation). The resulting glycosides have anomeric purity. By a cyclic process in which the nuclear molecule is treated continuously with a solution of glycosidase and donor sugar it is possible to achieve approximately quantitative conversion. Glycosidases having a broad donor specificity are usable in the form of a combinatory batch synthesis. A nuclear molecule is reacted e.g. with a glycosidase and a mixture of differing donor sugars. In this case, a saccharide library is obtained whose composition is determined inter alia by the specificity of the enzyme and the reactivity of the donor sugars. The person skilled in the art knows the processes suitable for the enzymatic binding of saccharides to nuclear molecules and materials necessary for this purpose.

Saccharide libraries according to the invention distinguish themselves by providing a plurality of differing saccharide-containing molecules. Furthermore, saccharide libraries according to the invention, particularly the nuclear molecules thereof, are resistant to degradation caused by glucosidase.

Therefore, saccharide libraries according to the invention are perfectly suited for a screening method by means of which specific active substances can be fished out of the saccharide library. In this connection, the following steps can be taken: In the development of an active substance on a saccharide basis, which reacts e.g. specifically with a known receptor, affinity chromatography will be applied, for example. For this purpose, the known receptor is immobilized, e.g. at a solid phase. By the application of the saccharide library to this solid phase only those saccharide-containing molecules are retained which bind to the receptor. All of the other saccharide-containing molecules are separated. Thereafter, all binding saccharide-containing molecules are eluted, e.g. by increasing the salt concentration of the solvent, and then analyzed. It can be favorable to already consider the analysis during the library synthesis. This can be done e.g. by not using a complete library, as described above, but obtaining a grouping of differing partial libraries by a clever division of the synthesis scheme, which grouping is then used. Partial libraries can be obtained e.g. in the following way: According to the above described process, the linkage to components A, B and C is carried out separately after the selective cleavage of a protecting group (R₁). Thus, three pots result, each differing by the first saccharide. Each of these three pots is processed further, but separately. At the end, three different partial libraries are obtained which can be used separately for screening. Depending on the pot containing the most active substance, the corresponding partial library can be shown again but in a further differentiated manner. In this way, structural evidence for the most active substance can be furnished.

### BRIEF DESCRIPTION OF THE DRAWING

FIG. 1 shows preferred nuclear molecules,

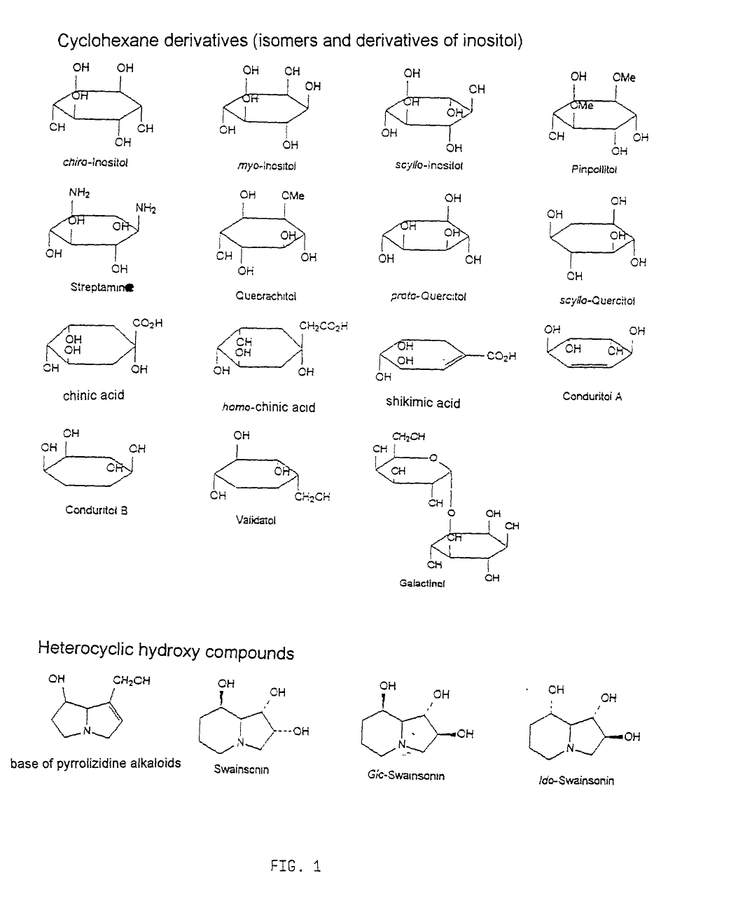

FIG. 2 shows the production of a saccharide library having a triamine as nuclear molecule,

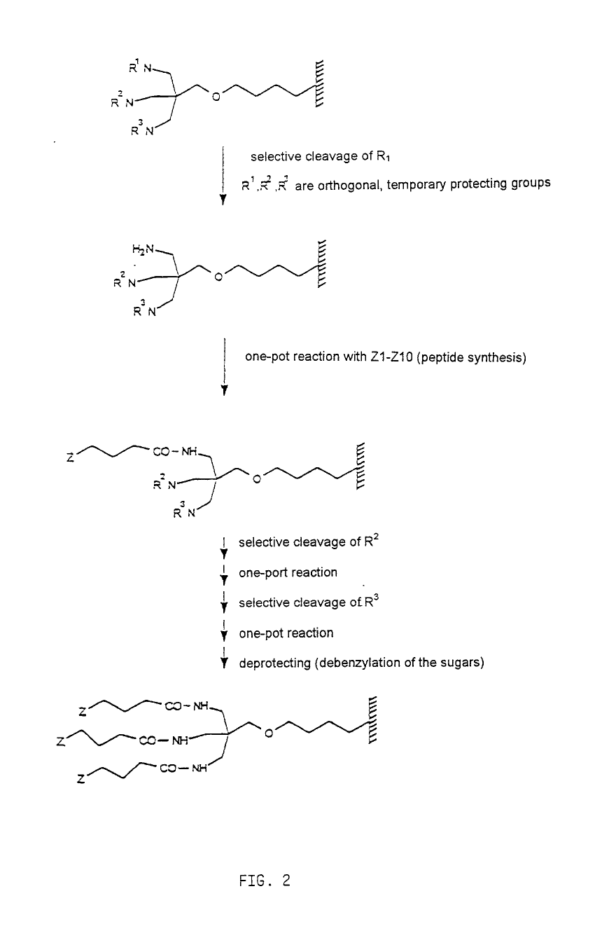

FIG. 3 shows the production of a saccharide background library, and

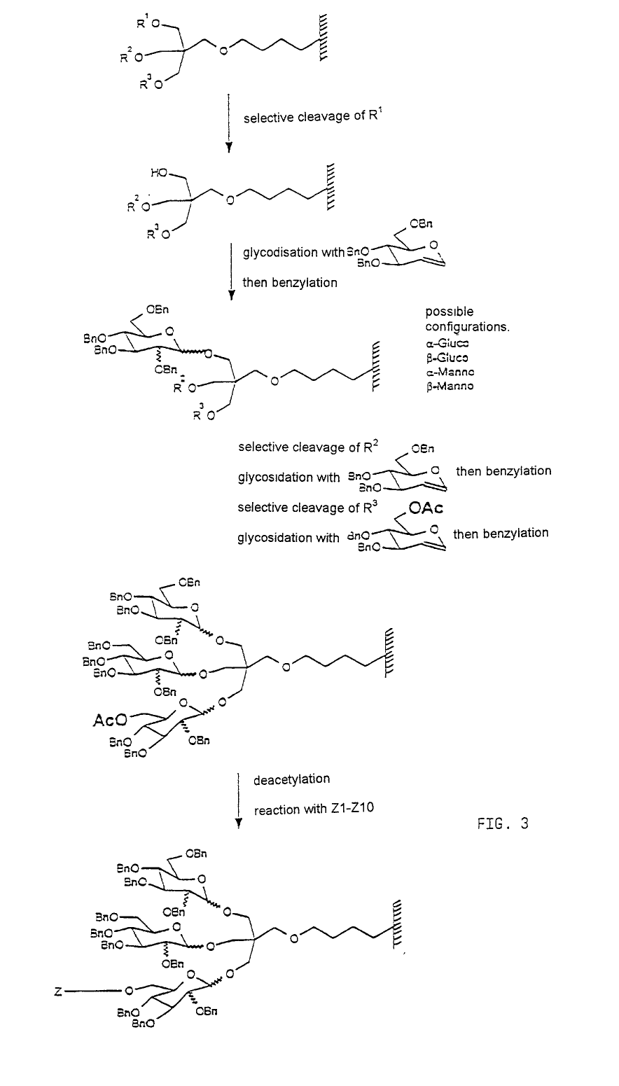

FIG. 4 shows the production of a saccharide library with an inositol as nuclear molecule.

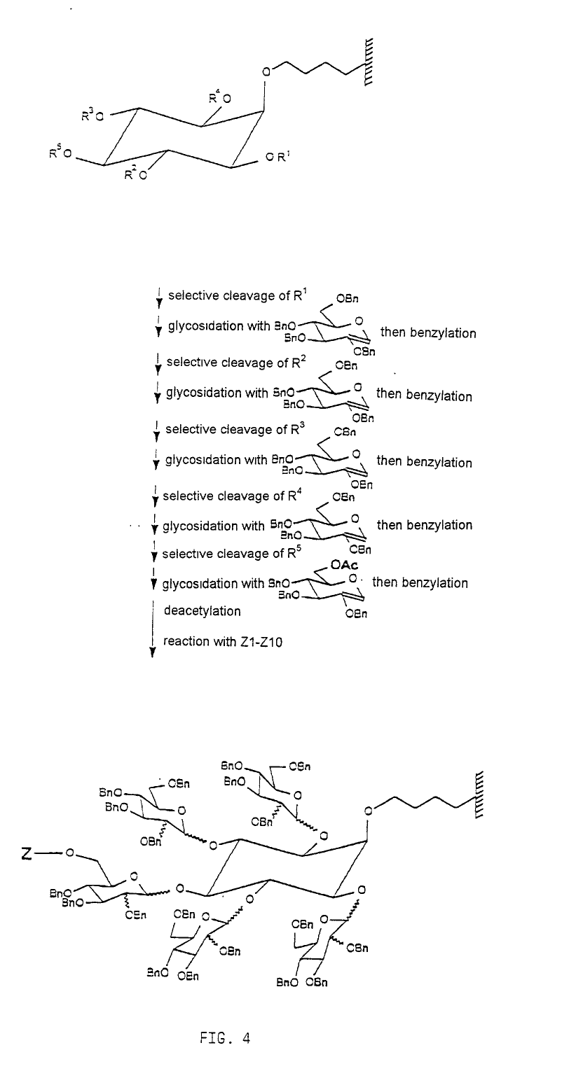

### ENCLOSURE 1

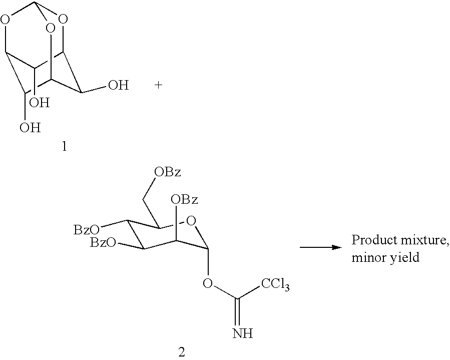

The core molecule is reacted with activated spacers 3 and 6.

### ENCLOSURE 2a

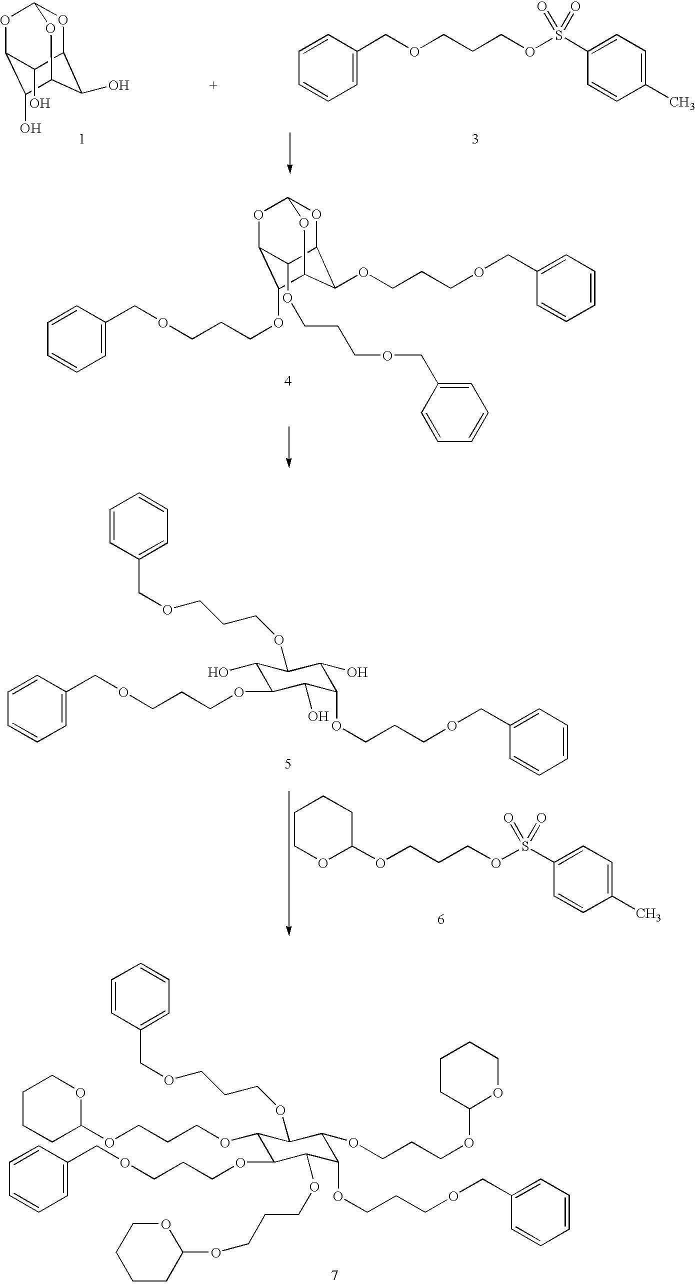

Thus, the hydroxy groups released after splitting off the protecting groups in 8 and 10, respectively, are much more free as regards access. The glycosidation of these hydroxy groups is accompanied by very high yields. At the same time, this also offers the possibility of providing the spacers with selectively releasable protecting groups 7. Thus, certain spacers can be provided in well-calculated fashion with defined saccharide units. The formation of the hexasaccharide 11 from trisaccharide 10 is described as an example of a linkage with a core molecule.

### ENCLOSURE 2b

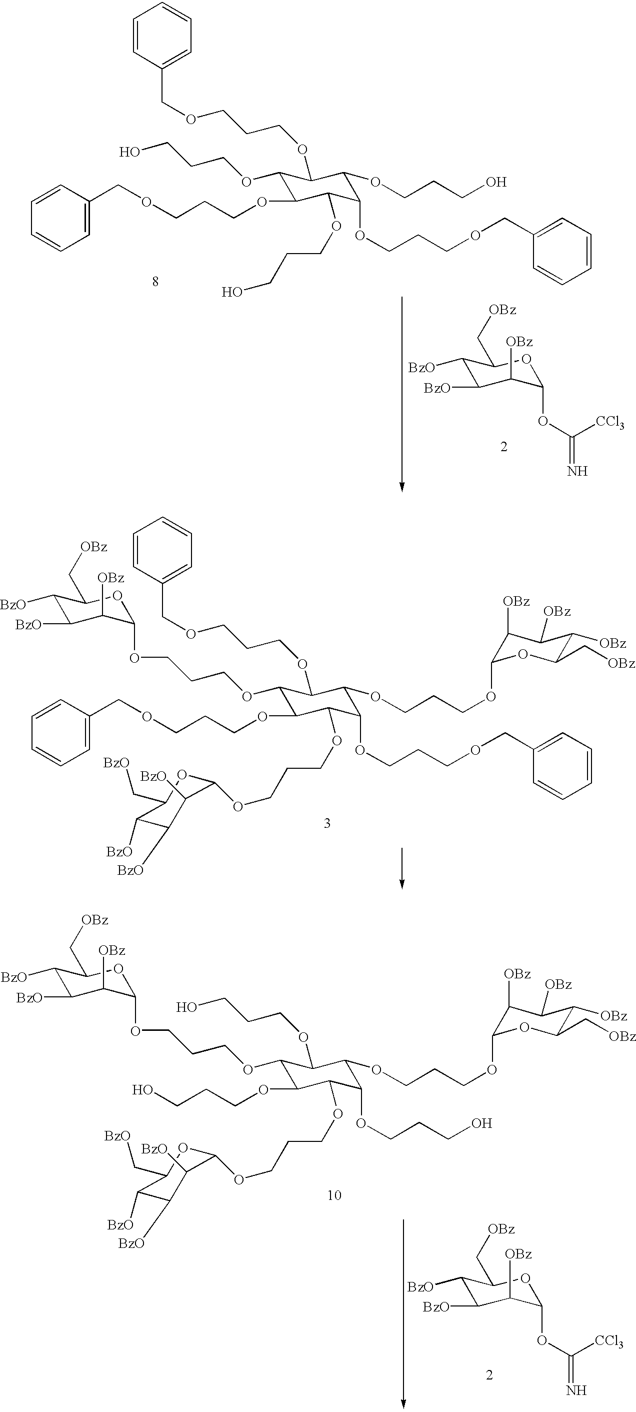

### ENCLOSURE 2c

754 mg triol 10 is stirred in 50 ml absolute dichloromethane with 1 g molecular sieve powder and the benzyl-protected imidate 2 in the presence of argon at room temperature for 30 min. The mixture is cooled down to −30° C. and slowly (for 10 min.) admixed with 50 μl trimethylsilyltriflate, dissolved in 10 ml absolute dichloromethane. The mixture is allowed to slowly reach a temperature of −20° C. Since in the thin layer chromatogram (silica gel: eluent petroleum ether-acetic ethyl ester 1:1 v/v, RF product=0.4) relatively much 10 glycosidated only once and twice can still be detected after 30 min., further 400 mg 2 are added and stirring is continued at −20° C. Cooling down to −30° C. then takes place, 50 μl triethylamine is added, put on the 50 ml NaHCO₃ solution and washed two times with NaHCO₃ solution in the separating funnel. The organic phase is dried with sodium sulfate and concentrated by rotating. Column chromatographic separation on silica gel: eluent petroleum ether-acetic ethyl ester 2:1 v/v, yields 1.34 g white foam 11=100% yield.

The linkage yield with respect to hexasaccharide is QUANTITATIVE!

By splitting off the protecting groups the desired saccharide 12 is obtained eventually.

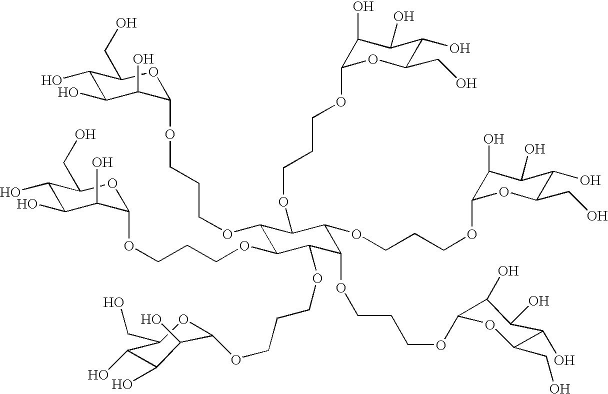

1. A saccharide library with various saccharide-containing molecules, in which each of the molecules comprises a nuclear molecule with at least two functional groups and at least two saccharides, a spacer being present between the nuclear molecule and one to maximally all saccharides.

2. The saccharide library according to claim 1, characterized in that the nuclear molecule is a cyclic aliphatic compound.

3. The saccharide library according to claim 2, characterized in that the cyclic aliphatic compound is a C₆ or C₅ cycloalkane.

4. The saccharide library according to claim 3, characterized in that the C₆ cycloalkane is a trihydroxycyclohexane, an inositol or a derivative thereof.

5. The saccharide library according to any one of claims 1 to 4, characterized in that the functional groups are hydroxy groups, amino groups, carboxylic acid groups, metallo-organic groups and/or halide groups.

6. The saccharide library according to any one of claims 1 to 5, characterized in that the saccharides are monosaccharide, disaccharide, trisaccharide and/or oligosaccharide, an inositol and/or a derivative thereof.

7. The saccharide library according to claim 6, characterized in that the monosaccharide is glucose or mannose.

8. The saccharide library according to any one of claims 1 to 7, characterized in that the saccharide-including molecule has 3, 4, 5 or 6 saccharides.

9. The saccharide library according to any one of claims 1 to 8, characterized in that the saccharides are equal or differ from one another.

10. The saccharide library according to any one of claims 1 to 9, characterized in that the spacer is an aliphatic compound.

11. The saccharide library according to any one of claims 1 to 10, characterized in that the spacer has 3 to 10 C atoms.

12. The saccharide library according to any one of claims 1 to 11, characterized in that the saccharide-containing molecule includes an organic compound.

13. The saccharide library according to claim 12, characterized in that the organic compound is an alkane having a functional group and/or an alkene.

14. The saccharide library according to claim 13, characterized in that the functional group is a halogen, a hydroxy, azido and/or amino group.

15. The saccharide library according to any one of claims 12 to 14, characterized in that the organic compound includes 3 to 10 C atoms.

16. The saccharide library according to any one of claims 12 to 15, characterized in that several organic compounds are present.

17. A process for the preparation of a saccharide library according to any one of claims 1 to 16, characterized in that the nuclear molecule, the saccharides, the spacer and optionally the organic compound are covalently bonded to one another.

18. Use of a saccharide library according to any one of claims 1 to 17 or detecting active substances against target proteins.

19. Use according to claim 18, wherein the target proteins are receptors.
